(19) United States
(12) Patent Application Publication
(10) Pub. No.: US 2010/0293626 A1
(43) Pub. Date: Nov. 18, 2010

(54) METHODS FOR IMPROVEMENT OF BIRTH RATES IN CANIDAE ON SOMATIC CELL NUCLEAR TRANSFER

(75) Inventors: Byeong Chun Lee, Seoul (KR); Min Kyu Kim, Seoul (KR); Goo Jang, Seoul (KR); Hyun Ju Oh, Seoul (KR); So Gun Hong, Seoul (KR); Jung Eun Park, Seoul (KR); Jung Taek Kang, Seoul (KR)

Correspondence Address: MOORE & VAN ALLEN PLLC, P.O. BOX 13706, Research Triangle Park, NC, 27709, US

(73) Assignee: SEOUL NATIONAL UNIVERSITY INDUSTRY FOUNDATION, Seoul (KR)

(21) Appl. No.: 12/517,567

(22) PCT Filed: Nov. 19, 2008
(86) PCT No.: PCT/KR08/06821

§ 371 Date: Mar. 2, 2010

(30) Foreign Application Priority Data
Nov. 19, 2007 (KR) 10-2007-0118164

(51) Int. Cl. C12N 15/06

(52) U.S. Cl. 800/21

(57) ABSTRACT

The present invention relates to a method for increasing the efficiency of offspring production in producing animals belonging to the family Canidae (canines) by somatic cell nuclear transfer. More specifically, relates to a method for increasing the efficiency of production of cloned canines by a method for cloning canines comprising enucleating the oocyte of a canine to prepare an enucleated oocyte, fusing a nuclear donor cell with the enucleated oocyte to prepare a nuclear transfer embryo and transferring the nuclear transfer embryo into the oviduct of a surrogate mother, wherein the nuclear donor cell is cultured in a medium containing a specific cell cycle synchronization-inducing substance such as roscovitine in the preparation thereof. The method enables to clone canines with high efficiency, and thus can contribute to the development of studies in the fields of veterinary medicine, anthropology and medical science such as the propagation of superior canines, the conservation of rare or nearly extinct canines, xenotransplantation and disease animal models.

### TECHNICAL FIELD

The present invention relates to a method for increasing the efficiency of offspring production in producing animals belonging to the family Canidae (canines) by somatic cell nuclear transfer (SCNT), and more particularly to a method for increasing the efficiency of production in cloned canids by SCNT. It comprises enucleating of the canine oocytes and fusing it with a nuclear donor cell to make a nuclear transfer embryo, and transferring it into the oviduct of a surrogate mother. The nuclear donor cell is cultured in the presence of a specific cell cycle synchronization-inducing substance such as roscovitine during its preparation thereof.

### BACKGROUND ART

Recently, with the development of biotechnology or genetic engineering, successful examples of the production of various recombinant organisms obtained by incorporating desired characteristics into useful crops have been reported. Recently, a number of successful examples of the production of cloned animals such as sheep have been reported. The production of cloned animals is actually possible only after highly accumulated technologies in the biotechnology field are preceded and thus can be considered as a barometer of relevant technological level.

SCNT technology, which is the technology allowing a living offspring to be born without undergoing meiosis and haploid germ cell retention which generally occurs in a generative process, is a method for development of new individuals by transferring diploid somatic cells of adults into enucleated oocytes to produce embryos and then transferring the embryos in vivo.

Generally, in the SCNT technology, recipient oocytes to be transferred with somatic cell are used after they are artificially cultured in vitro to metaphase II of meiosis. Then, in order to prevent the development of chromosomal abnormality resulting from SCNT, the mature oocytes are enucleated before transferring somatic cells. After injecting somatic cells into the perivitelline space or cytoplasm of the mature oocytes, the enucleated oocytes and the somatic cells are physically fused with each other by electrical stimulation. The fused couplets are activated by electrical stimulation or chemical substances, and then transferred into surrogate mothers to produce living offspring

Such SCNT technology can be widely used in the field, for example, in the propagation of superior animals, conservation of rare or nearly extinct animals, production of certain nutrients, production of therapeutic bio-materials, production of animals for organ transplantation, production of animals with diseases or disorders and production of animals medically suitable for alternative treatments to organ transplantation such as gene therapy.

The technology for cloning mammals by somatic cell nuclear transfer was first accomplished by Dr. Wilmut of the Roslin Institute, England, by taking a mammary gland cell from a six-year old sheep, transferring the cell into an enucleated oocyte to prepare a nuclear transfer embryo, and transferring the embryo in vivo, thus producing a cloned animal, Dolly. Since then, cloned cows, mice, goats, pigs and rabbits have been produced by nuclear transfer using the somatic cells obtained from adult animals (WO 99/37143A2, EP 930009A1, WO 99/34669A1, WO 99/01164A1 and U.S. Pat. No. 5,945,577).

Meanwhile, not only the cloning of industrial animals, such as cows and pigs, but also the cloning of pet animals such as dogs, attract the interest of many persons. Recently, among pet animals, a cat was first cloned, and a study on dog cloning was also conducted using millions of dollars in research funding.

However, it is very difficult to retrieve immature oocytes from the ovary of a female dog that is not in heat, and culture the oocytes in vitro to develop mature oocytes. This is one of reasons why the cloning of dogs by somatic cell nuclear transfer is very difficult compared to other animals due to the unique species-specific reproductive characteristics of dogs. Nevertheless, dogs have physiological characteristics similar to those of humans and have disease development patterns similar to those of humans, and thus humans and dogs are similar to each other in pathological and physiological terms. The number of inherited disorders which can be actually used in human disease research is 224 in dogs, which is significantly larger than 65 in pigs or 136 in cats (http://omia.angis.org.au/). Thus, if such genetic characteristics are used to produce cloned dogs for studying human disease models, the cloned dogs will be greatly helpful in human disease research. For this reason, attempts to clone dogs have been made for a long period of time, but the success rate of dog cloning is still very low, and the dog cloning is considered to be difficult to succeed. Thus, there is a need to develop an effective method for improving the efficiency of cloning of canines.

Accordingly, the present inventors have conducted studies on an improved method for producing cloned canines using the SCNT method and, as a result, have found that cloned canines can be produced with high efficiency by a method in which a specific substance such as roscovitine is added during the preparation of nuclear donor cells, thereby completing the present invention.

### SUMMARY OF INVENTION

It is an object of the present invention to provide a method for producing a canine nuclear transfer embryo, and a method for increasing the efficiency of production of cloned offspring, using SCNT technology.

Another object of the present invention is to provide a canine nuclear transfer embryo produced by said method.

Still another object of the present invention is to provide a method for producing a cloned canine, with increased production efficiency, the method comprising a step of transferring said nuclear transfer embryo into a surrogate mother to produce a living offspring.

To achieve the above objects, the present invention provides a method for producing a canine nuclear transfer embryo using SCNT technology, wherein the nuclear transfer embryo is produced using nuclear donor cells synchronized in a specific cycle through a process of culturing in the presence of a cell cycle synchronization-inducing substance such as roscovitine in the preparation of the nuclear donor cells.

The present invention also provides a canine nuclear transfer embryo produced according to said method.

The present invention also provides a method for producing a cloned canine, with increased efficiency, the method comprising a step of transferring said nuclear transfer embryo into a surrogate mother to produce a living offspring.

Other features and embodiments of the present invention will be more apparent from the following detailed description and the appended claims

### BRIEF DESCRIPTION OF DRAWINGS

FIG. 1 is photographs showing the nuclear remodeling of a cloned embryo constructed with a nuclear donor cell treated with Roscovitine. In FIG. 1, white arrows indicate nuclear shapes in various stages.

FIG. 2 is a photograph (FIG. 2(a)) of seven 1-month-old Retriever dogs cloned according to the inventive method, a photograph (FIG. 2(b)) taken when the dogs were 4-month-old, and a photograph (FIG. 2(c)) of a dog produced by SCNT.

FIG. 3 is a photograph (FIG. 3(a) of four cancer-sniffing dogs produced according to the inventive method and a photograph (FIG. 3(b)) of five cloned Pit Bull terrier dogs produced, according to the inventive method.

### DETAILED DESCRIPTION OF THE INVENTION AND PREFERRED EMBODIMENTS

The definition of terms used herein is as follows.

The term “nuclear transfer” as used herein refers to a gene manipulation technique allowing an identical characteristics and qualities acquired by artificially combining an enucleated oocytes with a cell or a nucleus of cell.

The term “nuclear transfer embryo” as used herein refers to an embryo injected or fused with a nuclear donor cell.

The term “cloned (or cloning)” as used herein refers to a gene manipulation technique for preparing a new individual unit to have a gene set identical to another individual unit. Particularly, in the present invention, the term “cloned” is used herein to mean that a cell, an embryonic cell, a fetal cell, and/or an animal cell have a nuclear DNA sequence which is substantially similar or identical to the nuclear DNA sequence of another cell.

The term “nuclear donor cell” as used herein refers to a cell or a nucleus of cell which is transferred into a recipient oocyte as a nuclear acceptor.

As used herein, the term “subculture” refers to a method for re-culturing the cells in a new dish after separating the adherent cell which reached confluency from the former culture dish to prevent overgrowth. Specifically, it refers to a method for isolating cells from animals and continuously subjecting the cells to primary culture, secondary culture, tertiary culture, etc., that is, a method for preserving cell lines by periodically replacing with fresh media.

The term “recipient oocyte” as used herein refers to an oocyte that receives a nucleus from a nuclear donor cell after removing its original nucleus.

The term “oocyte” as used herein refers to a mature oocyte which has reached metaphase II of meiosis. The term “enucleated oocyte” as used herein refers to an oocyte which its nucleus has been removed.

The term “fusion” as used herein refers to a combination of a nuclear donor cell and a lipid membrane of a recipient oocyte. For example, the lipid membrane may be the plasma membrane or nuclear membrane of a cell. Fusion may occur upon application of an electrical stimulus between a nuclear donor cell and a recipient oocyte when they are placed adjacent to each other or when a nuclear donor cell is placed in a perivitelline space of a recipient oocyte.

The term “activation” as used herein refers to stimulation of a cell to divide, before, during or after nuclear transfer. Preferably, in the present invention, it means stimulation of a cell to divide after nuclear transfer.

The term “living offspring” as used herein means an animal that can survive ex utero. Preferably, it is an animal that can survive for one second, one minute, one day, one week, one month, six months or more than one year. The animal may not require an in utero environment for survival.

In the present invention, animals belonging to the family canidae (canines) can be broadly divided into Tribe Canini and Tribe Vulpini, and include dogs, wolves, foxes, jackals, coyotes, Korean wolves and raccoon dogs. Preferably, they include dogs or wolves. The dogs are known to result from the domestication of wild wolves, and thus, wolves and dogs have the same chromosome number and show similarity in gestation period and sex hormone changes (Seal, U S et al., Biology Reproduction, 21:1057-1066, 1979). In the present invention, the term “animals belonging to the family canidae is also simply abbreviated as “canines”.

In one aspect, the present invention relates to a method for producing a canine nuclear transfer embryo by somatic cell nuclear transfer technology.

Specifically, the inventive method for producing a canine nuclear transfer embryo comprises the steps:
(a) removing the nucleus from canine oocytes to prepare an enucleated oocyte;
(b) preparing a nuclear donor cell by culturing somatic cells in the presence of a cell cycle synchronization-inducing substance when culturing the somatic cells derived from the tissue of canids;
(c) microinjecting the nuclear donor cell of the step (b) into the enucleated oocyte of the step (a) and fusing between the donor cell and the enucleated oocyte; and
(d) activating the fused oocyte of the step (c).

Particularly, step (b) of the method for producing the canine nuclear transfer embryo is characterized in that the somatic cell isolated from canine tissue is cultured in the presence of the cell cycle synchronization-inducing substance. The cell cycle synchronization-inducing substance is a substance that temporarily arrests cells in any one phase of the cell cycle consisting of meiotic phase (M), the early phase of DNA synthesis (G1 phase), the DNA synthesis phase (S phase) and the late phase of DNA synthesis (G2 phase), and when the substance is removed, the cell cycle arrested at a specific cycle will progress again. By adding the cell cycle synchronization-inducing substance as described above, the efficiency of production of canine offspring by somatic cell nuclear transfer can be increased.

Examples of the cell cycle synchronization-inducing substance include: roscovitine (Formula 1) as a Cdk (cyclin-dependent kinase) inhibitor blocking the G0/G1 phase; cycloheximide (Formula 2) blocking the G0/G1 phase; dimethyl sulfoxide (DMSO) (Formula 3) blocking the G0/G1 phase; butyrolactone I (Formula 4) as a Cdk inhibitor blocking the G1/S phase; aphidicolin (Formula 5) as an inhibitor of DNA polymerase A,D blocking the early S phase; demecolcine (Formula 6) blocking the M phase in mitotic metaphase; mimosine (Formula 7) as a DNA replication inhibitor blocking the S phase; colchicines (Formula 8) as a microtubule inhibitor blocking the G2/M phase; and Hoechst 33342 (Formula 9) as DNA topoisomerase, and the chemical formula of each of the substances is as follows. Preferred is roscovitine, cycloheximide or DMSO, and the most preferred is roscovitine.

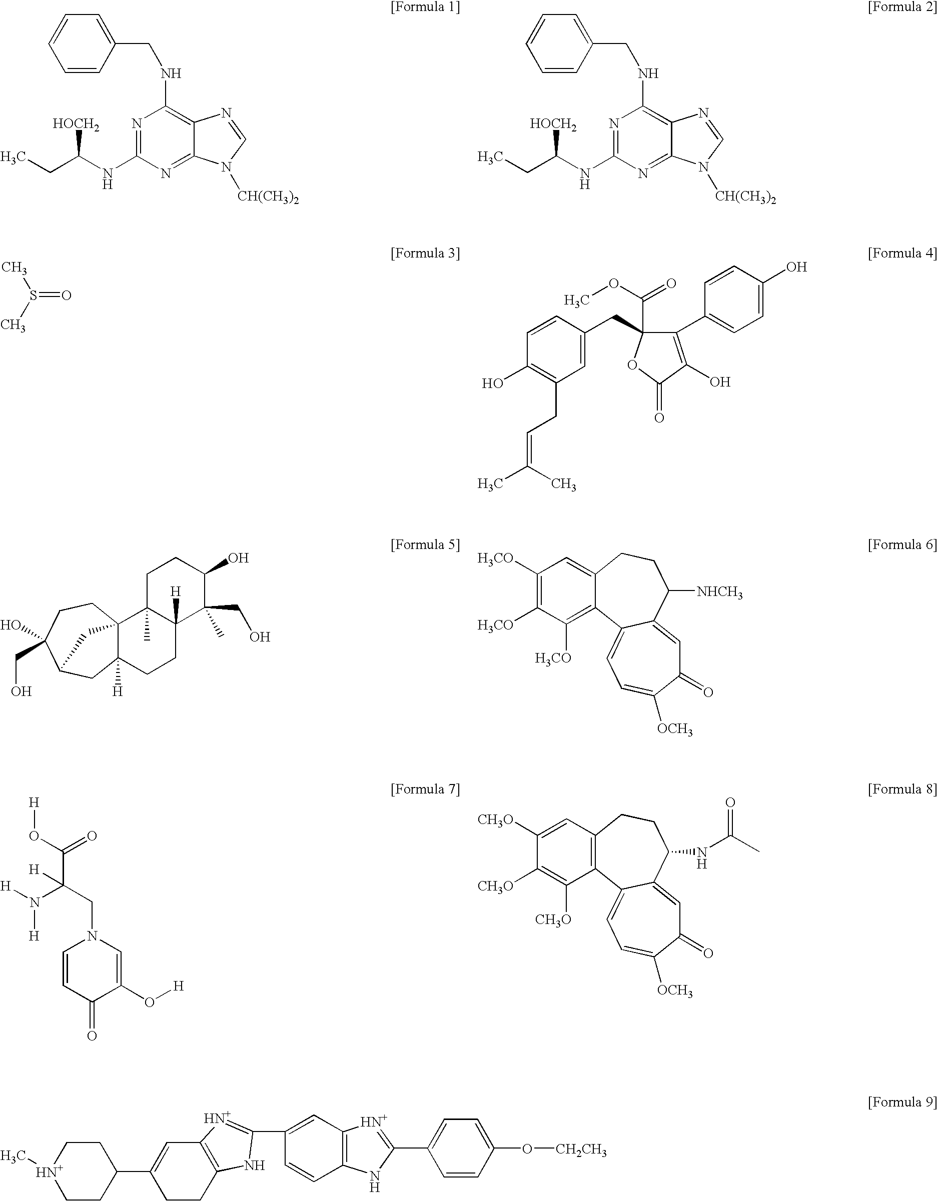

In one embodiment of the present invention, a nuclear donor cell may be prepared by adding roscovitine to a somatic cell isolated from canine tissue, and then culturing the cell. When the efficiencies of production of cloned dogs according to the addition and non-addition of roscovitine are compared, the pregnancy rate of a control group not treated with roscovitine is only 10%, whereas a group treated with roscovitine shows a pregnancy rate of about 40%, suggesting that the addition of roscovitine in the preparation of a nuclear donor cell significantly increases the production rate of cloned dogs. Thus, the present invention includes a nuclear transfer embryo prepared according to the above-described method.

In another aspect, the present invention relates to a method for producing a cloned canine, characterized in that said nuclear transfer embryo is used.

More specifically, the inventive method for producing a cloned canine comprises the steps of:
(a) removing the nucleus from canine oocytes to prepare an enucleated oocyte;
(b) preparing a nuclear donor cell by culturing somatic cells in the presence of a cell cycle synchronization-inducing substance when culturing the somatic cells derived from the tissue of canids;
(c) microinjecting the nuclear donor cell of the step (b) into the enucleated oocyte of the step (a) and fusing between the donor cell and the enucleated oocyte; and
(d) activating the fused oocyte of the step (c); and
(e) transferring the activated oocyte into the oviduct of a surrogate mother.

In the step (b) of the inventive method for producing a cloned canine, in order to increase the efficiency of production of a canine offspring by somatic cell nuclear transfer, the nuclear donor cell is prepared by adding the cell cycle synchronization inducing substance such as roscovitine, cyclohesimide, DMSO, butyrolactone I, aphidicolin, demecolcine, mimosine, colchicine, Hoechst 33342 or the like to the somatic cell isolated from the canine tissue, and then culturing the somatic cell. The specific description of the above substances is as described above.

The inventive method for producing a canine nuclear transfer embryo and the inventive method for producing a cloned canine will now be described in detail.

### Step 1: Enucleation of Recipient Oocytes

Generally, the oocytes of mammals (e.g., cattle, pigs and sheep) are ovulated in mature oocytes, i.e., metaphase II of meiosis, whereas canine oocytes are ovulated at prophase I of meiosis unlike other animals and matured while staying in the oviduct for 48-72 hours.

Recipient oocytes may be canine immature oocytes, mature oocytes, and oocytes undergoing initial aging, moderate aging and severe aging. Preferably, for use as recipient oocytes, immature oocytes collected from canines can be matured in vitro, or oocytes matured in vivo can be collected. Because the in vitro maturation rate of canine oocyte nucleus is very low and the ovulation time and reproductive physiology of canines are different from other animals, it is preferable to collect canine oocytes matured in vivo for use as recipient oocytes. More specifically, the collection of mature oocytes from canines is preferably conducted at 48-72 hours and more preferably 72 hours after ovulation induction in the canines.

In this regard, the day of ovulation in canines can be determined by any method known in the art. Examples of the method for determining the day of ovulation include, but are not limited to, vaginal smear tests, the measurement of serum sex hormone levels, and the use of ultrasonographic diagnosis systems. The start of estrus in canines can be confirmed by vulva swelling and serosanguinous discharge. In one Example of the present invention, vaginal smear test and the analysis of serum progesterone concentration were conducted. As a result, the day on which nonkeratinized epithelial cells reached more than 80% and serum progesterone concentration initially reached more than about 4.0 ng/mL was regarded as the day of ovulation.

As a method for collecting oocytes matured in vivo, a surgical method including anesthetizing an animal followed by laparotomy can be used. More specifically, the collection of oocytes matured in vivo can be performed using salpingectomy known in the art. The salpingectomy is a method comprises surgically excising the oviduct, flushing an oocyte collection medium into the oviduct and collecting oocytes from flushing solution.

In another method, oocytes matured in vivo can be collected by inserting a catheter into the fimbriated end of the oviduct, and injecting a flushing solution into the uterotubal junction using an indwelling needle. This method has an advantage in that it does not cause damage to the oviduct, and thus allows an oocyte donor animal to be used for the next estrus.

After the collection of mature oocytes, the haploid nuclei of the oocytes are removed. The enucleation of the oocytes can be performed using any method known in the art (U.S. Pat. No. 4,994,384, U.S. Pat. No. 5,057,420, U.S. Pat. No. 5,945,577, EP 0930009A1, KR 10-0342437, Kanda et al., J. Vet. Med. Sci., 57(4):641-646, 1995; Willadsen, Nature, 320:63-65, 1986, Nagashima et al., Mol. Reprod. Dev., 48:339-343, 1997; Nagashima et al., J. Reprod Dev., 38:37-78, 1992; Prather et al., Biol. Reprod., 41:414-418, 1989, Prather et al., J. Exp. Zool., 255:355-358, 1990; Saito et al., Assis Reprod Tech Andro, 259:257-266, 1992; Terlouw et al., Theriogenology 37:309, 1992).

Preferably, the enucleation of recipient oocytes can be performed by either of the following two methods. One method comprises removing a cumulus cell of a mature recipient oocyte, partially dissecting the zona pellucida of the recipient oocyte using a microneedle by making a slit, and removing the first polar body, nucleus and cytoplasm (in the smallest amount possible) through the slit. Another method comprises removing a cumulus cell of a mature recipient oocyte, staining the oocyte, and removing the first polar body and nucleus of the oocyte using an aspiration pipette. More preferably, for the enucleation of oocytes, the aspiration method is used for oocytes with high viability, whereas the method for forming a slit is used for oocytes with low viability, which is decided by visual examination of the recipient oocytes.

### Step 2: Preparation of Nuclear Donor Cells

In the production of transgenic animals expressing a target gene by somatic cell nuclear transfer technology, nuclear donor cells are required. As nuclear donor cells in the present invention, somatic cells derived from canines are used. Specifically, somatic cells used in the present invention may be canine embryonic cells, fetal derived cells, juvenile cells, or adult derived cells, preferably adult derived cells originated from tissue such as the cumulus, skin, oral mucosa, blood, bone marrow, liver, lungs, kidneys, muscles and reproductive organs, etc.

Examples of somatic cells which can be used in the present invention include, but are not limited to, cumulus cells, epithelial cells, fibroblasts, neural cells, keratinocytes, hematopoietic cells, melanocytes, chondrocytes, erythrocytes, macropharges, monocytes, muscle cells, B lymphocytes, T lymphocytes, embryonic stem cells and embryonic reproductive cells. More preferably, somatic cells which can be used in the present invention may include fetal fibroblasts, adult fibroblasts, and cumulus cells. Most preferably, fibroblasts isolated from canine fetuses and adults are used. These cells have advantages in that a large number of the cells can be obtained in the initial isolation stage, culture of the cells is relatively easy, and in vitro culture and manipulation of the cells are easy.

The somatic cells which are provided as the nuclear donor cells can be obtained by a method for preparing surgical samples or biopsy samples, and from the samples, single cells cultured in optimized conditions can be obtained using the following method.

Tissue is collected from an animal subject, and cells are isolated from the tissue. Then, the cells are cultured in a tissue culture basal medium, and a cell cycle synchronization-inducing substance is added thereto, followed by the culture of the cells. When the cells completely grow, the cells are collected by treatment with trypsin, and then can be used as nuclear donor cells.

The present invention is characterized in that a cell cycle synchronization-inducing substance is added in order to increase the efficiency of production of cloned canines. Cell cycle synchronization-inducing substances which can be used in the present invention include: roscovitine (Formula 1) as a Cdk (cyclin-dependent kinase) inhibitor blocking the G0/G1 phase; cycloheximide (Formula 2) blocking the G0/G1 phase; dimethyl sulfoxide (DMSO) (Formula 3) blocking the G0/G1 phase; butyrolactone I (Formula 4) as a Cdk inhibitor blocking the G1/S phase; aphidicolin (Formula 5) as an inhibitor of DNA polymerase A,D blocking the early S phase; demecolcine (Formula 6) blocking the M phase in the mitotic metaphase; mimosine (Formula 7) as a DNA replication inhibitor blocking the S phase; colchicines (Formula 8) as a microtubule inhibitor blocking the G2/M phase; and Hoechst 33342 (Formula 9) as DNA topoisomerase, and the chemical formula of each of the substances is as follows. Preferred is roscovitine, cycloheximide or DMSO, and the most preferred is roscovitine. In one embodiment, tissue from an animal to be cloned is aseptically dissected to obtain a surgical sample or a biopsy sample, and the sample is minced, treated with trypsin and then cultured in tissue culture medium. As the tissue culture medium, those known in the art may be used and examples thereof include TCM-199, DMEM (Dulbecco's modified Eagle's medium) and the like.

After culturing for 3-4 days in the tissue culture medium, the growth of the cells on a culture dish is confirmed. When the cells completely grow, the cells are treated with trypsin, some of the tissue is frozen and stored in liquid nitrogen for later use, and the remnants are subcultured for use in nuclear transfer. The cells to be continuously cultured for use in nuclear transfer are subcultured in a fresh culture dish, and then treated with trypsin to prepare single cells for use in nuclear transfer.

Finally, roscovitine is added to the cells, which are then additionally cultured. Then, the cells are collected by treatment with trypsin and subjected to somatic cell nuclear transfer. Herein, the concentration of roscovitine added is preferably 5-30 μM, and more preferably 10-20 μM, and the culture time is preferably 18-72 hours, and more preferably 24-48 hours.

In one embodiment, when the concentration of cells after culture in a fresh culture dish reaches about 60%, the cells are treated with 15 μM of roscovitine for 18-24 hours, and then treated with trypsin to prepare single cells. Then, the cells are used in nuclear transfer.

### Step 3: Microinjection and Fusion of Nuclear Donor Cells—Preparation of Nuclear Transfer Embryos

The nuclear donor cells prepared in the step 2 are microinjected into the enucleated oocytes prepared in the step 1. Herein, the microinjection is performed by microinjecting the nuclear donor cells between the cytoplasm and zona pellucida of the enucleated oocytes using a transfer pipette.

The enucleated oocytes microinjected with nuclear donor cells are electrically fused with nuclear donor cells using a cell manipulator. The electrical fusion can be performed with direct current or alternating current. Preferably, it can be performed at a voltage of 2.0-6.0 kV/cm, and more preferably, it can be performed 1-3 times at a direct current voltage of 3.0-5.0 kV/cm for 10-30 μs. Most preferably, direct current is applied twice each at a voltage of 3.5-5.0 kV/cm for 15 μs.

The above-described voltage range in the electrical fusion is characterized in that it is much higher than a voltage range in general electrical fusion known until now. This range is an optimized condition for electrical fusion and allows the production of cloned canines with a higher cloning efficiency.

The fusion of the nuclear donor cell to the oocyte by electrical stimulation can be carried out in various fusion media, for example, Zimmerman or mannitol. Preferably, a medium containing mannitol, MgSO₄, Hepes and BSA can be used.

After the enucleated oocytes are subjected to somatic cell nuclear transfer, and then electrical fusion, the nuclei of the oocytes undergo a remodeling process. As evidence indicating that the remodeling smoothly occurred, premature chromosome condensation (PCC) occurs in the fused embryos at about 1 hour after electrical fusion. It is known that the fused embryos which underwent the premature chromosome condensation are easily developed and reprogrammed after remodeling and activation.

In the case in which nuclear donor cells treated with a cell cycle synchronization-inducing substance such as roscovitine have been transferred, PCC occurs at a rate higher than that in the case in which the nuclear donor cells have not been transferred. Also, the continued swelling and recondensation of nuclei occur even after the activation of the fused embryos (see Test Example 1 of the present invention). Particularly, PCC (premature chromosome condensation), NE (nuclear enlargement) and NS (nuclear swelling), which are processes that nuclei undergo with the passage of time during the normal development thereof, occur in the order of PCC→NE→NS with the passage of time. In cloned embryos obtained by transferring nuclear donor cells treated with a cell cycle synchronization-inducing substance such as roscovitine, PCC occurs at significantly high rate, and thus the nuclear remodeling is more activated.

### Step 4: Activation of Nuclear Transfer Embryos

Activation of fused nuclear transfer embryos is a step of reactivating a cell cycle temporarily arrested in the maturation process. For this purpose, it is necessary to reduce the activity of cell signal delivery materials such as MPF, MAP kinase etc., which are factors of cell recycle arrest.

Generally, methods of activating the nuclear transfer embryos include an electrical method and a chemical method. In one embodiment of the present invention, the chemical method may be used to activate nuclear transfer embryos. The chemical method can promote the activation of nuclear transfer embryos according to the present invention more than the electrical method. The chemical methods include a method for treating nuclear transfer embryos with substances such as ethanol, inositol trisphosphate, bivalent ions (e.g., Ca²⁺ or Sr²⁺), microtubule inhibitors (e.g., cytochalasin B), bivalent ion ionophores (e.g., Ca²⁺ ionophore ionomycin), protein kinase inhibitors (e.g., 6-dimethylaminopurin), protein synthesis inhibitors (e.g., cycloheximide) or phorbol 12-myristate 13-acetate.

Preferably, as the chemical method for the activation of nuclear transfer embryos, a method for treating the nuclear transfer embryos simultaneously or stepwise with calcium ionophore and 6-dimethylaminopurin can be used in the present invention. More preferably, the nuclear transfer embryos are treated with 5-10 μM calcium ionophore at 37-39° C. for 3-6 minutes and then with 1.5 mM-2.5 mM 6-dimethylaminopurin at 37-39° C. for 4-5 hours.

In another aspect, the present invention relates to canine nuclear transfer embryos prepared according to the above-described method.

Step 5: Transfer of Nuclear Transfer Embryos into Surrogate Mother and Production of Living Offspring

Furthermore, the canine nuclear transfer embryos may be used to produce cloned canines by transferring them into surrogate mothers to allow living offspring to be born.

In the case of canines, nuclear transfer embryos are transferred immediately after activation without in vitro culture. The transfer can be performed by any method known in the art, and preferably, a catheter can be used to transfer the cloned embryos.

Surrogate mothers suitable for the transfer of the nuclear transfer embryos and capable of developing the embryos into normal fetuses are selected. The best time for the transfer is determined by monitoring estrus and ovulation of either canines showing natural estrus after reaching maturity or canines before or after sexual maturity, the estrus of which has been induced by artificial hormone treatment. Generally, the suitable transfer time may be consistent within the range of 1-2 days with the ovulation day of a canine oocyte donor which provided oocytes used in nuclear transfer. Preferably, the transfer time is one day after the ovulation day of the canine oocyte donor, and most preferably, the same day as the ovulation day of the canine oocyte donor. The preferred evaluation of the estrus cycle of a surrogate mother may be based on the concentration of progesterone.

Transfer of the nuclear transfer embryos into a surrogate mother is performed by transferring the embryos into the oviducts of the surrogate mothers by laparotomy. In the transfer of the nuclear transfer embryos into the surrogate mother, the nuclear transfer embryos may preferably be at the 1-cell, 2-cell or 4-cell stage. For this purpose, the transfer of the nuclear transfer embryos into the surrogate mothers is preferably performed within 4 hours after activation. Also, the nuclear transfer embryos can be cultured in 25 0 microdrops of mSOF covered with mineral oil until surrogate mothers are prepared, then transferred into the surrogate mother.

3 weeks after embryo transplantation, the surrogate mothers are evaluated for pregnancy by ultrasound. After that, the ultrasonic diagnosis is carried out every two weeks to monitor the pregnancy of the surrogate mother and the growth state of fetuses.

If living offspring are not delivered even after the delivery interval exceeds 30 min, an experienced assistant should help delivery of a surrogate mother. When the expected delivery date is passed, the delivery of offspring is induced by injecting a hormone preparation into the surrogate mother, or by surgical operation such as Caesarean section.

The inventive method for the production of cloned canines has the effect of increasing the pregnancy success rate which was very low in the prior art. Accordingly, the inventive method can overcome the shortcoming of the previously known method which was difficult to use in practice due to low cloning efficiency, and thus it can be practically applied to increase the efficiency of production of cloned canines.

### EXAMPLES

Hereinafter, the present invention will be described in further detail with reference to examples. It is to be understood, however, that these examples are for illustrative purposes only and are not to be construed to limit the scope of the present invention.

### Example 1

### Collection of Recipient Oocytes from Dogs

Dogs used as oocyte donors in the experiment were 1-5-year-old female dogs which showed a regular estrus cycle and had no disease in the reproductive organs. The dogs used as oocyte donors were kept according to the standards established by the Seoul National University for Accreditation of Laboratory Animal Care. Ovulation day was determined by performing a vaginal smear test and measuring serum progesterone concentration every day in estrus dogs showing the natural estrus. Also, mature oocytes were surgically retrieved at 72 hours after ovulation.

In order to measure serum progesterone concentration, 3-5 ml of blood was collected everyday and centrifuged to obtain serum, and the serum was analyzed using a DSL-3900 ACTIVE Progesterone Coated-Tube Radioimmunoassay Kit (Diagnostic Systems Laboratories, Inc., USA). The day on which the progesterone concentration initially reached more than 4.0 ng/ml was considered as the day of ovulation (Hase et al., J. Vet. Med. Sci., 62:243-248, 2000).

To perform the vaginal smear test, smears were obtained daily from the day of the initial sign of proestrus. Smears were collected by inserting a swab into the lips of the vulva and rolled on a slide glass. After staining with a Diff-Quik staining (International Chemical Co., Japan), the smears were examined with a microscope; the time at which superficial cells reached more than 80% of the epithelial cells (cornified index, Evans, J. M. et al., Vet. Rec., 7:598-599, 1970) was regarded as the time of ovulation.

The present inventors determined the time of ovulation according to the above-described method, and then retrieved oocytes from donor dogs by laparotomy in the following manner.

First, female dogs as oocyte donors were anesthetized by administering 6 mg/kg of ketamine HCl and 1 mg/kg of xylazine. The anesthesia was maintained by administering isoflurane.

A needle having a rounded front end was inserted into the fimbriated end of the oviduct of the anesthetized dog through the bursal slit. The inserted needle was held in place with suture. Herein, a quick-release device including a 3 cm plastic tube (2 mm diameter) and hemostatic forceps was used. To make the tube of the oviduct well visible, digital pressure was applied to the oviduct and the surrounding uterus-oviduct junction, and an intravenous catheter (24 gauge) was inserted. Then, Hepes-buffer as an oocyte collection medium shown in Table 1, which contains 10% (v/v) FBS, 2 mM NaHCO₃ and 5 mg/ml BSA (Invitrogen, Carlsbad, Calif.), was flushed through the catheter to allow oocytes to flow out.

TABLE 1

| Component | Content | |
|---|---|---|
| TCM powder 1 L (Gibco 31100-027) | 9.9 | g |
| P/S antibiotic | 1%(penicillin 10000 IU, | |
| | streptomycin 10 mg) | |
| HEPES buffer | 2.38 | g |
| FBS | 10% | (v/v) |
| NaHCO₃ | 0.1680 | g |
| BSA | 5 | mg/L |

### Example 2

### Enucleation of Recipient Oocytes

The oocytes obtained in Example 1 were added to the oocyte collection medium of Table 1, and cumulus cells were removed from the oocytes by repeatedly pipetting hyaluronidase (Sigma, USA) in the medium. Then, the oocytes from which cumulus cells have been removed were stained with 5 μg/mL Hoechst 33342 for 5 minutes and observed under an inverted microscope at 200× magnification so as to select only oocytes having first polar body extruded.

The selected oocytes were enucleated using a micromanipulator (Narishige, Tokyo, Japan) in the above medium (Table 1) supplemented with 5 μg/mL cytochalasin B. Specifically, the oocytes were held with a holding micropipette (about 150 μm diameter), and then the first polar body, adjacent cytoplasm (less than 5%) and oocyte nuclei were removed using an aspiration pipette (about 20 μm diameter). The enucleated oocytes were stored in a TCM-199 medium (Table 2) supplemented with 10% (v/v) FBS.

TABLE 2
TCM-199 medium

| Component | Content | |
|---|---|---|
| TCM199 liquid | 89 | ml |
| pyruvic acid | 0.0099 | g |
| P/S(antibiotic) | 1 | ml |

| | |
|---|---|
| FBS | 10% |

### Example 3

### Preparation of Nuclear Donor Cells

As nuclear donor cells, adult fibroblasts collected from dogs were used. For this purpose, an ear skin biopsy was taken from dogs. Small pieces of the ear tissue fragment were washed three times with DPBS (Dulbecco's Phosphate Buffered Saline) and minced with a surgical blade. The minced tissue was added to 1 mM EDTA-containing DMEM (Dulbecco's modified Eagle's medium) (DMEM Life Technologies, Rockville, Md.) and centrifuged at 300×g for 2 minutes. Then, the cells were seeded into 60 mm plastic culture dishes (Becton Dickinson, Lincoln Park, N.J.).

Then, the cells were cultured for 3-4 days in DMEM supplemented with 10% (v/v) FBS, 1 mM glutamine, 25 mM NaHCO₃ and 1% (v/v) minimal essential medium (MEM) nonessential amino acid solution (Invitrogen, CA) at 39° C. in a humidified atmosphere of 5% CO₂ and 95% air.

After the cells were cultured to confluency, unattached cells were removed, and the remaining attached cells were treated with trypsin in a medium supplemented with 0.1% trypsin and 0.02% EDTA for 1 minute and were further subcultured at 4-6-10 day intervals in three fresh culture dishes. Then, the subcultured cells were placed in a freezing medium consisting of 80% (v/v) DMEM, 10% (v/v) DMSO and 10% (v/v) FBS and were stored in liquid nitrogen at −196° C.

Before performing somatic cell nuclear transfer, the cells were thawed and cultured for 24 hours in a medium supplemented with 15 μM roscovitine (i.e., DMEM+10% FBS+15 μM roscovitine). Then, the cells were treated with trypsin for about 2 minutes during somatic cell nuclear transfer and collected from the monolayer.

### Example 4

### Somatic Cell Nuclear Transfer

The nuclear donor cells prepared in Example 3 were microinjected into the enucleated oocytes prepared in Example 2. The nuclear donor cells were microinjected into a perivitelline space of the enucleated oocytes in the following manner. The enucleated oocytes were treated with 100/mL phytohemagglutinin in the medium of Table 1, and the slit of the enucleated oocytes was held with a holding pipette. Then, a transfer pipette was inserted into the slit, and the single cells isolated from fibroblasts in Example 3 were injected between the cytoplasm and zona pellucida of the enucleated oocytes by the transfer pipette.

Then, the nuclear donor cell-oocyte couplets were placed in a fusion medium (containing 0.26 M mannitol, 0.1 mM MgSO₄, 0.5 mM HEPES and 0.05% BSA) and interposed between two parallel electrodes attached to a micromanipulator (Nikon-Narishige, Japan). Then, the fusion of the couplets was induced by electrical stimulation applied twice at a voltage of 4 kV/cm for 15 μs using an electro-cell fusion apparatus (NEPA GENE Co., Chiba, Japan).

After 1 hour of electrical stimulation, the fusion of the nuclear donor cells with the oocyte cytoplasm was observed under a stereomicroscope. The fused embryos were selected and cultured for 1.5-4 hours in TCM-199 (Table 2) supplemented with 10% (v/v) FBS.

### Example 5

### Activation of Nuclear Transfer Embryos

The nuclear transfer embryos obtained in Example 4 were cultured in mSOF containing 10 μM ionophore (Sigma) at 39° C. for 4 minutes, thus inducing the activation of the nuclear transfer embryos. Then, the nuclear transfer embryos were washed and further cultured in mSOF (Table 3) supplemented with 1.9 mM 6-dimethylaminopurin for 4 hours.

The nuclear transfer embryos were cultured in 25 μl microdrops of mSOF overlaid with mineral oil, before they were transferred into surrogate mothers.

TABLE 3

| Component | | | | Concentration | | Volume | |
|---|---|---|---|---|---|---|---|
| NaCl(54.44) | 2.900-3.100 | g/ml | Stock-T | 107.7 | mM(3.14 g) | 2 | ml |
| Kcl(74.55) | 0.2669 | g | | 7.2 | mM | | |
| KH₂PO₄ (136.1) | 0.0810 | g | | 1.2 | mM | | |
| Sod Lactate | 0.28 | ml | | 3.3 | mM | | |
| Kanamycin | 0.0375 | g | | | | | |
| Phenel-Red | 0.0050 | g | | | | | |

| | | | | | | |
|---|---|---|---|---|---|---|
| NaHCO₃(84.01) | 1.0531 g/50 ml | Stock-B | 25.1 | mM | 2 | ml |
| | 0.42124 g/20 ml | | | | | |
| Sod. Pyruvate(110.0) | 0.0182 g/5 ml | Stock-C | 0.3 | mM | 200 | μl |
| MgCl₂6H₂O(147.0) | 0.0996 g/10 ml | Stock-M | 0.5 | mM | 200 | μl |
| CaCl₂2H₂O(203.3) | 0.2514 g/10 ml | Stock-D | 1.71 | mM | 200 | μl |
| Glucose(180) | 0.27024 g/10 ml | | 1.5 | mM | 200 | μl |
| Glutamine(146.1) | 0.14618 g/10 ml | | 1 | mM | 200 | μl |
| Citri Acid(192) | 0.096 g/10 ml | Stock-CA | 0.5 | mM | 200 | μl |
| HEPES(238.3) | 0.5958 g/10 ml | Stock-E | 2.5 | mM | 200 | μl |

| | | | | |
|---|---|---|---|---|
| EAA(Gibco 11051-018) | | | 400 | μl |
| NEAA(Gibco 11140-019) | | | 200 | μl |
| ITS(I-3146) | | | 100 | μl |
| BSA(fatty acid free) | | | 0.1600 | g |
| Hyaluronic Acid | 0.5 | mg/ml | | |
| 1N NaOH | | | | |
| D.W. | | | total 20 | ml |

pH 7.2-7.4/osmotic pressure 275-285/EAA, NEAA: sensitive to light

### Example 6

### Embryo Transfer into Surrogate Mothers and Production of Cloned Dogs

The nuclear transfer embryos from Example 5 were surgically transferred into the oviduct of estrus synchronized surrogate mothers. The transfer was carried out within 4 hours after the activation of the nuclear transfer embryos. As the surrogate mothers, dogs were used, which were disease-free, showed the repetition of the normal estrus cycle and had a normal uterine condition. For the transfer, the surrogate mothers were anesthetized by vascular injection with 0.1 mg/kg acepromazine and 6 mg/kg propofol, and maintained at the anesthetized state using 2% isoflurane. Operation area of the anesthetized female dogs was aseptically operated and incised on the center of the abdomen according to general laparotomy so as to expose the oviduct. The abdominal cavity was stimulated by hand to draw the ovary, the oviduct and the uterus to the incision. The mesovarium of the drawn ovary was carefully handled to recognize the opening of the oviduct, and a 3.5 F Tom cat catheter (Sherwood, St. Louis, Mo.) equipped with a 1.0 ml tuberculin syringe (Latex free, Becton Dickinson & CO. Franklin lakes, N.J. 07417) was inserted into the oviduct to secure a sufficient space in the front of the catheter. Then, the nuclear transfer embryos were injected into the oviduct through the catheter. Whether the nuclear transfer embryos were successfully injected was observed under a microscope. The abdominal suture was performed with an absorbable suture material, and then, skin suture was performed. To prevent post-surgery infection, a broad range of antibiotic was injected for 3 days.

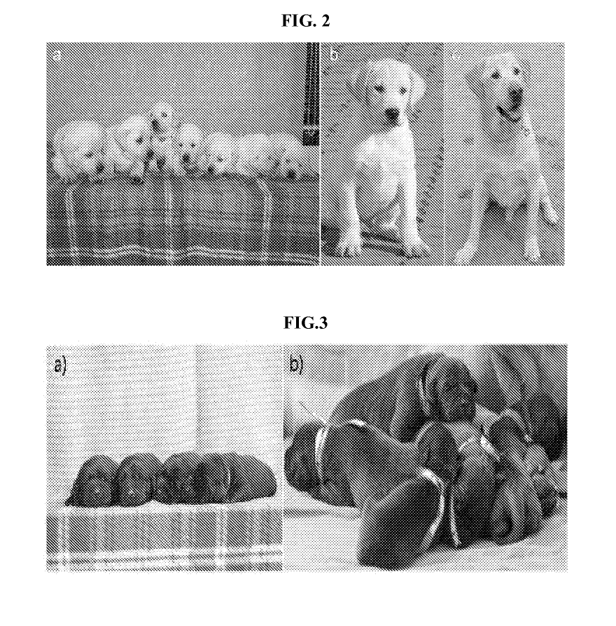

At 23 days after transferring the nuclear transfer embryos into the surrogate mothers, pregnancies were detected using a SONOACE 9900 (Medison Co. LTD, Seoul, Korea) ultrasound scanner with an attached 7.0 MHZ linear probe. Pregnancy was monitored by ultrasound every 2 weeks after initial confirmation. As a result, it was confirmed that the dogs had become pregnant, and the cloned dogs were produced by inducing natural delivery or performing Cesarean section. The cloned dogs are shown in FIGS. 2 and 3.

### Test Example 1

### Comparison Between Group Treated with Roscovitine and Control Group

After performing canine somatic cell nuclear transfer into the enucleated oocytes using the microinjection method, the difference in the formation and change of nuclei between a control group not treated with roscovitine and a group treated with roscovitine for 24 hours, was examined.

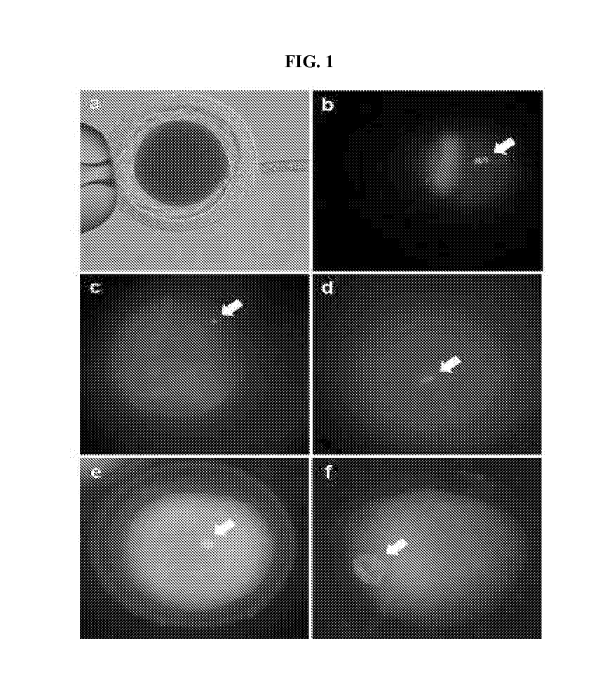

As a result, it could be seen that, in the group treated with roscovitine, premature chromosome condensation (PCC) occurred at a rate higher than that in the control group, and the continued swelling and recondensation of nuclei occurred even after the activation of the fused embryos while showing a significant difference from the control group. Such results indicate that the reconstructed cloned embryos of dogs can be normally remodeled even after the nuclei of canine oocytes are substituted with the microinjection method and that the treated group develops in a more excellent manner compared to the control group. Various morphological patterns of nuclei resulting from such nuclear remodeling are shown in Table 4 and FIG. 1.

Table 4 shows the comparison of nuclear remodeling of reconstructed oocytes between the roscovitine-treated group and the control group after transferring somatic cells into enucleated oocytes. In Table 4, PCC, NE and NS indicate phenomena that nuclei undergo with the passage of time during the normal development thereof, and such processes occur in the order of PCC→NE→NS with the passage of time. It was observed that, at 1 hour after electrical fusion, PCC occurred in the treated group at a rate significantly higher than that in the control group. Also, it was observed that, at 4 hours after the activation of the fused embryos, nuclear swelling (NS) occurred more in the treated group than in the control group.

TABLE 4

| | | No. of | | | | | |
|---|---|---|---|---|---|---|---|
| Time | | nuclear | No. of | | | | |
| (hpf/ | | transfer | reconstructed | For reconstructed oocytes | | | |
| hpa) | Treat | embryos | oocytes | IN (%) | PCC (%) | NE (%) | NS (%) |
| 1 hpf | Control | 32 | 27 | 24 | 3 | 0 | 0 |
| | | | | (88.8 ± 7.9) | (11.1 ± 7.9) | | |
| | Test | 39 | 34 | 11 | 23 | 0 | 0 |
| | group | | | (32.3 ± 7.0) | (67.6 ± 7.0) | | |
| 4 hpa | Control | 36 | 30 | 0 | 2 | 17 | 11 |
| | | | | | (6.6 ± 3.7) | (56.6 ± 8.4) | (36.6 ± 8.5) |
| | Test | 45 | 38 | 0 | 1 | 9 | 28 |
| | group | | | | (2.6 ± 3.3) | (23.6 ± 7.5) | (73.6 ± 7.5) |

(hpf = hour post fusion; hpa = hour post activation; IN = intact nucleus; PCC = premature chromosome condensation; NE = nuclear enlargement; NS = nuclear swelling. (P < 0.05))

Then, a group of nuclear donor cells treated with roscovitine according to the inventive method and a control group of nuclear donor cells cultured without treatment with roscovitine were subjected to somatic cell nuclear transfer, and then the pregnancy rate of each of the groups was examined.

As a result, in the control group, 478 embryos transferred with somatic cell nuclei were transferred into 26 surrogate mothers, and among them, 4 animals became pregnant (15.3%, the number of pregnant surrogate mothers/the total number of surrogate mothers). In the group treated with roscovitine, 556 embryos after somatic cell nuclear transfer were transferred into 29 surrogate mothers, and among them, 11 animals succeeded in pregnancy (39.9%, the number of pregnant surrogate mothers/total number of surrogate mothers).

From such results, it could be seen that pregnancy rate was very significantly increased when somatic cell nuclear transfer was performed after treatment with roscovitine.

TABLE 5

| | No. of | No. of | No. of | Pregnancy rate | Pregnancy rate |
|---|---|---|---|---|---|
| | transfer | surrogate | pregnant | (based on surrogate | (based on transfer |
| Treatment | embryos | mothers | mothers | mothers) | embryos) |
| Control | 478 | 26 | 4 | 15.38% | 1.040% |
| Group | 556 | 29 | 11 | 39.93% | 3.95% |
| treated with | | | | | |
| roscovitine | | | | | |

### INDUSTRIAL APPLICABILITY

As described above in detail, the method according to the present invention can increase the efficiency of successful nuclear transfer in canine cloning by inducing the cell cycle synchronization of donor cells for nuclear transfer using a specific substance. Accordingly, the inventive method can contribute to the development of studies in the fields of veterinary medicine, anthropology and medical science such as the propagation of superior canines, the conservation of rare or nearly extinct canines, xenotransplantation and disease animal models.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof

What is claimed is:

1. A method for producing a canine nuclear transfer embryo comprises the steps:
preparing an enucleated oocyte; preparing a nuclear donor cell; microinjecting the nuclear donor cell and then electrically fusing the nuclear donor cell with the enucleated oocyte; and activating the fused oocyte,
wherein the step of preparing a nuclear donor cell comprises culturing the nuclear donor cell in the presence of a cell cycle synchronization-inducing substance selected from the group consisting of roscovitine, cyclohesimide, DMSO, butyrolactone I, aphidicolin, demecolcine, mimosine, colchicine, Hoechst 33342 when culturing the nuclear donor cell derived from the tissue of canids.

2. The method for producing a canine nuclear transfer embryo according to claim 1, wherein the cell cycle synchronization-inducing substance is roscovitine.

3. The method for producing a canine nuclear transfer embryo according to claim 1, wherein the cell cycle synchronization-inducing substance is added at a concentration of 5˜30 μM.

4. The method for producing a canine nuclear transfer embryo according to claim 1, wherein the said culturing of the nuclear donor cell by addition of the cell cycle synchronization-inducing substance is performed for 18˜72 h.

5. The method for producing a canine nuclear transfer embryo according to claim 1, wherein the nuclear donor cell is selected from the group consisting of cumulus cell, epithelial cell, fibroblast, neural cell, keratinocyte, hematopoietic cell, melanocyte, chondrocyte, erythrocyte, macropharge, monocyte, muscle cell, B lymphocyte, T lymphocyte, embryonic stem cell, embryonic germ cell, fetal cell, placenta cell, and adult cell.

6. The method for producing a canine nuclear transfer embryo according to claim 5, wherein the nuclear donor cell is a fibroblast or a cumulus cell.

7. A method for producing a cloned canine comprises the steps: preparing an enucleated oocyte; preparing a nuclear donor cell; microinjecting the nuclear donor cell and then electrically fusing the nuclear donor cell with the enucleated oocyte; activating the fused oocyte and transferring the activated oocyte into the oviduct of a surrogate mother, wherein the step of preparing a nuclear donor cell comprises culturing the nuclear donor cell in the presence of a cell cycle synchronization-inducing substance selected from the group consisting of roscovitine, cyclohesimide, DMSO, butyrolactone I, aphidicolin, demecolcine, mimosine, colchicine, Hoechst 33342 when culturing the nuclear donor cell derived from the tissue of canids.

8. The method for producing a cloned canine according to claim 7, wherein the cell cycle synchronization-inducing substance is roscovitine.

9. The method for producing a cloned canine according to claim 7, wherein the cell cycle synchronization-inducing substance is added at a concentration of 5˜30 μM.

10. The method for producing a cloned canine according to claim 7, wherein said culturing the nuclear donor cell by addition of the cell cycle synchronization-inducing substance is performed for 18˜72h.

11. The method for producing a cloned canine according to claim 7, wherein the nuclear donor cell is selected from the group consisting of cumulus cell, epithelial cell, fibroblast, neural cell, keratinocyte, hematopoietic cell, melanocyte, chondrocyte, erythrocyte, macropharge, monocyte, muscle cell, B lymphocyte, T lymphocyte, embryonic stem cell, embryonic germ cell, fetal cell, placenta cell, and adult cell.

12. The method for producing a cloned canine according to claim 11, wherein the nuclear donor cell is a fibroblast or a cumulus cell.
